(19) United States
(12) Patent Application Publication
(10) Pub. No.: US 2009/0299118 A1
(43) Pub. Date: Dec. 3, 2009

(54) FCC For Light Feed Upgrading

(75) Inventor: Alan Claude, Houston, TX (US)

Correspondence Address: KELLOGG BROWN & ROOT LLC;ATTN: Christian Heausler, 4100 Clinton Drive, HOUSTON, TX, 77020, US

(73) Assignee: KELLOGG BROWN & ROOT LLC, Houston, TX (US)

(21) Appl. No.: 12/128,961

(22) Filed: May 29, 2008

(51) Int. Cl. C07C 4/06

(52) U.S. Cl. 585/653

(57) ABSTRACT

Systems and methods for upgrading hydrocarbons are provided. A first hydrocarbon can be cracked in the presence of one or more catalysts to provide a first cracked mixture containing one or more light cycle oils (LCOs) and one or more coked catalysts. A second hydrocarbon, containing one or more C4 to C20 hydrocarbons and having a Research Octane Number of less than 88, can be mixed with the one or more catalysts to provide a first mixture at a second temperature. A third hydrocarbon can be combined with the first mixture to provide a second mixture. The second mixture can be cracked to provide a second cracked mixture containing propylene, one or more mixed hydrocarbons in the gasoline boiling range having a Research Octane Number greater than 88, and one or more coked catalysts. The first cracked mixture and second cracked mixture can be combined to provide a third mixture.

### BACKGROUND

1. Field

The present embodiments generally relate to systems and methods for refining hydrocarbons. More particularly, embodiments of the present invention relate to systems and methods for upgrading hydrocarbons to provide olefins, cycle oils, and high-octane blendstocks.

2. Description of the Related Art

Fluid Catalytic Crackers (FCCs) offer tremendous operational flexibility in converting one or more hydrocarbon feeds into a variety of finished products. Surprisingly, FCCs, for their great flexibility, are relatively simple, often containing one or more riser reactors, one or more disengagers and one or more regenerators. The finished product matrix from an FCC will depend upon a variety of factors including the operating conditions within the FCC (e.g. temperature, pressure, and catalyst to oil ratio), the residence time of the hydrocarbons within the FCC, and the cracking catalyst used within the FCC.

Light olefins, primarily ethylene and propylene, are produced using typical FCC feedstocks in an FCC operating at high severity conditions—high riser outlet temperature and/or high catalyst to oil ratio. Byproducts include high octane naphtha suitable for gasoline blending (typically C₅ to C₁₂ hydrocarbons) and cycle oils (C₁₂ and higher hydrocarbons). A catalyst additive, such as ZSM-5, is often added to enhance production of lighter olefins. High riser outlet temperatures (in excess of 540° C.) and high catalyst to oil ratios (often 20:1 or higher) can produce high coke yields. Depending on feedstock properties, burning the coke off the catalyst to reactivate it can produce excessive temperatures resulting in a permanent loss of catalyst activity. While excessive temperatures can be controlled by adding a coolant such as water to generated steam, using this excess heat to crack other feeds to the FCC can prove beneficial.

The high octane gasoline blending stock yield can be increased by operating at less severe conditions such as a riser outlet temperature between 520° C. and 540° C., and a catalyst to oil ratio of about 5:1 to about 12:1. The light olefins yield is typically lower and cycle oil yield typically higher for this lower severity mode of operation. The minimum octane required for blending the gasoline into the refinery gasoline pool often limits the operating conditions within the FCC. Frequently, either or both the riser outlet temperature and/or the catalyst to oil ratio must be increased to increase the octane value of the products, while at the same time decreasing gasoline yield. It might be possible to increase gasoline yield if some method for simultaneously upgrading the octane value of the products.

Cycle oils, including light cycle oils having between twelve and twenty carbon atoms (C₁₂ to C₂₀), and heavy cycle oils having more than twenty carbon atoms (C₂₀+) can be produced using an FCC wherein a hydrocarbon is cracked at relatively low severity operating conditions, for example at riser outlet temperatures of about 450° C. to about 520° C. Cycle oils can be further processed into one or more high-value finished products such as kerosene, diesel fuel, or other fuel oils. Frequently, kerosene and fuel oils can have a higher market value than olefins and naphthas removed from the kerosene and fuel oils during refining. Two competing factors, however, can limit the operational flexibility of an FCC producing light cycle oils for the kerosene and fuel oil market. First, the octane value of the naphthas removed from the cycle oil must be sufficient to permit the use of the naphthas as a gasoline blendstock. At times, this may require a cut in cycle oil yield as the FCC is operated at higher severity conditions to improve the octane rating of the naphthas. Secondly, operation at low severity conditions can reduce the quantity of coke formed on the catalyst. Since coke is used as a fuel, generating heat in the catalyst regeneration process, a reduction in coke on the catalyst can require the use of supplemental fuel for catalyst regeneration.

At least a portion of the light hydrocarbons, such as naphthas and other light hydrocarbons, separated from the cycle oil can be upgraded to provide one or more high-value finished products. For example, the naphthas and light hydrocarbons can be further cracked to provide one or more mixed hydrocarbons, including paraffins, olefins, naphthenes, and aromatics, suitable for use as a high-octane gasoline blendstock. The cracking of naphthas and other light hydrocarbons typically requires the use of specialized catalysts and high severity conditions, i.e. high temperature and/or catalyst to oil ratio, within the riser reactor.

Thus, a need exists for improved systems and methods for producing cycle oil within an FCC while providing an efficient process for upgrading naphthas and other light hydrocarbon byproducts generated during downstream processing of the cycle oil.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts an illustrative system for upgrading one or more hydrocarbons according to one or more embodiments described.

FIG. 2 depicts another illustrative system for upgrading one or more hydrocarbons according to one or more embodiments described.

### DETAILED DESCRIPTION

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions, when the information in this patent is combined with available information and technology.

Systems and methods for processing hydrocarbons are provided. A first hydrocarbon can be cracked at a first temperature in the presence of one or more catalysts to provide a first cracked mixture comprising one or more light cycle oils and one or more coked catalysts. A second hydrocarbon containing one or more C₄ to C₂₀ hydrocarbons having a Research Octane Number of less than 88 can be mixed or otherwise combined with the one or more catalysts to provide a first mixture at a second temperature. A third hydrocarbon can be introduced to the first mixture to provide a second mixture. The second mixture can be cracked at a third temperature to provide a second cracked containing one or more mixed hydrocarbons having a Research Octane Number greater than about 88 and one or more coked catalysts. The first cracked mixture and second cracked mixture can be combined to provide a third mixture.

The first hydrocarbon can include one or more heavy hydrocarbons having twenty or more carbon atoms (C₂₀+) including, but not limited to one or more demetallized oils, deasphalted oils, vacuum gas oils, fuel oils, gas oils, resids, mixtures thereof or any combination thereof. The first hydrocarbon can have a normal boiling point of about 200° C. (390° F.) or more; about 250° C. (480° F.) or more; about 300° C. (570° F.) or more; about 350° C. (660° F.) or more; or about 400° C. (750° F.) or more.

The second hydrocarbon can include, but is not limited to mixed C₄'s from other refining process such as cokers and visbreakers, light naphtha recycle from the products of the first hydrocarbon, naphtha products from other refining processes such as cokers and visbreakers, straight naphthas either produced in the refinery or imported from outside, straight run kerosene/diesel streams, mixtures thereof, derivatives thereof, or any combination thereof. In one or more embodiments, the second hydrocarbon can be selected to maximize the yield of light olefins with high octane gasoline blend stock as a byproduct. In one or more embodiments, the second hydrocarbon can be selected to improve the octane of the C₅ to C₁₂ segment of the product stream with one or more light olefins, including but not limited to ethylene, propylene, butylenes, or any combination thereof as byproducts.

The third hydrocarbon can include demetallized oils, deasphalted oils, vacuum gas oils, fuel oils, gas oils, resids having twenty or more carbon atoms (C₂₀+), mixtures thereof or any combination thereof (“heavy feeds” or “heavy hydrocarbons”). In one or more embodiments, the third hydrocarbon can have a similar or identical composition to the first hydrocarbon. In one or more embodiments, at least a portion of the first hydrocarbon, with or without the addition of one or more heavy hydrocarbons listed above, can be segregated to provide the third hydrocarbon. The third hydrocarbon can provide sufficient coke to maintain the required mixture temperatures of both the first hydrocarbon/catalyst system and the second hydrocarbon/third hydrocarbon/catalyst system.

In one or more embodiments, the third mixture can be selectively separated and at least a portion recycled to provide at least a portion of the second hydrocarbon. In one or more embodiments, one or more hydrocarbons containing twelve or fewer carbon atoms (C₁₂), for example one or more naphthenic hydrocarbons, can be selectively separated and recycled to provide at least a portion of the second hydrocarbon.

The one or more catalysts can include one or more catalysts useful for the production of cycle oil, one or more catalysts useful for improving the Research Octane Number of the cracked hydrocarbon compounds, one or more catalysts useful for improving the production of olefinic hydrocarbons, mixtures thereof, or any combination thereof. In one or more embodiments, the one or more catalysts can include one or more catalysts useful for improving the olefinic hydrocarbon concentration and increasing the Research Octane Number of the one or more cracked hydrocarbon compounds in the second cracked mixture.

The first temperature can be maintained anywhere from “low severity” conditions—i.e. low temperature and/or catalyst to oil ratio with low catalyst activity to “high severity” conditions—i.e. high temperature and/or high catalyst to oil ratio with high catalyst activity. Operation at low severity operations can increase the production of cycle oils, particularly light cycle oils. Low severity conditions can include a first hydrocarbon and catalyst mixture temperature of from about 455° C. (850° F.) to about 520° C. (970° F.). In one or more embodiments, at moderate severity conditions, for example at operating temperatures of about 520° C. to about 565° C., the second hydrocarbon can assist in upgrading the octane of the naphtha produced by cracking the first hydrocarbon to a Research Octane Number greater than 88. In one or more embodiments, at high severity conditions, for example at operating temperatures of from about 540° C. to about 700° C., the second hydrocarbon can assist in increasing the propylene production by re-cracking the naphtha and C₄'s produced by cracking the first hydrocarbon. In one or more embodiments, the third hydrocarbon can include one or more hydrocarbons originating external to the FCC, thereby improving the octane content or propylene production. In one or more embodiments, the third hydrocarbon can be used to heat balance that the FCC thereby ensuring proper regeneration of the FCC catalyst without the need for supplemental regeneration fuel.

In one or more embodiments, the temperature of the first hydrocarbon and catalyst can be at moderate severity, ranging from a low of about 520° C. (970° F.) to a high of approximately 540° C. (1,000° F.). At moderate severity conditions, the yield of one or more high-octane naphthas can be improved when compared to high-octane naphtha yield at low severity operation. In one or more embodiments, the Research Octane Number of the one or more high-octane naphthas can be greater than about 88. In one or more embodiments, the second hydrocarbon can contain one or more recycled C₄ hydrocarbons and/or one or more light naphthas. In one or more embodiments, the second hydrocarbon can be cracked at high severity conditions within the FCC to increase the propylene yield. In one or more embodiments, one or more hydrocarbons external to the FCC can be introduced to the FCC to improve the octane yield at moderate severity conditions, to improve propylene yields at high severity conditions, or any intermediate combination thereof A third hydrocarbon can be used to maintain the heat balance within the FCC where the first and second hydrocarbons are unable to provide sufficient coke build on the catalyst.

In one or more embodiments, the temperature of the first hydrocarbon and catalyst can be maintained at high severity conditions, ranging from a low of about 540° C. (1,000° F.) to a high of about 565° C. (1,050° F.). At high severity conditions, the olefin yield, including ethylene and propylene, can increase when compared to operation at less severe conditions. However, operating at high severity conditions can promote the formation of excessive coke on the FCC catalyst. When catalyst containing excessive coke is regenerated within the regenerator, the excessive coke can cause high regenerator temperatures. In one or more embodiments, the regenerator temperatures can be lowered by introducing a second hydrocarbon to the riser. The second hydrocarbon can include, but is not limited to one or more recycled C₄ hydrocarbons, one or more light naphthas, one or more hydrocarbons external to the FCC, or any combination thereof. In one or more embodiments, the second hydrocarbon can be cracked at high severity conditions favoring the production of light olefins, including but not limited to ethylene and propylene. In one or more embodiments, the second hydrocarbon can be cracked at moderate severity conditions favoring the production of high-octane blendstocks.

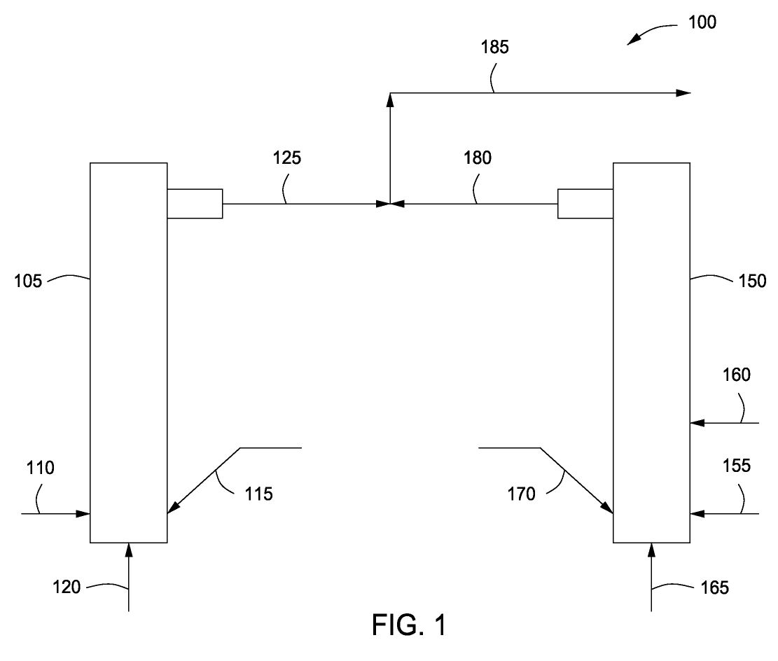

FIG. 1 depicts an illustrative system 100 for upgrading one or more hydrocarbons according to one or more embodiments. In one or more embodiments, the system 100 can include two or more riser reactors 105, 150. The hydrocarbon feed (“first hydrocarbon” or “first hydrocarbon feed”) in line 110 can be mixed or otherwise combined with one or more catalysts supplied via line 115, and optionally, steam supplied via line 120 in the first riser reactor 105. In one or more embodiments, the first hydrocarbon feed in line 110 can be partially or completely vaporized prior to introduction to the riser reactor 105. In one or more embodiments, the first hydrocarbon feed in line 110 can be about 1% wt. or more; about 5% wt. or more; about 10% wt. or more; about 25% wt. or more; about 50% wt. or more; about 75% wt. or more; or about 90% wt. or more vaporized prior to introduction to the riser reactor 105. The first hydrocarbon feed in line 110 can be introduced to the riser reactor 105 at ambient temperature or at an elevated temperature. In one or more embodiments, the temperature of the first hydrocarbon feed in line 110 can be a minimum of about 40° C. (105° F.); about 150° C. (300° F); about 260° C. (500° F.); or about 370° C. (700° F.).

The optional steam introduced via line 120 can be either saturated or superheated. In one or more embodiments, the steam introduced via line 120 can be saturated, having a minimum supply pressure of about 135 kPa (5 psig); about 310 kPa (30 psig); about 510 kPa (60 psig); about 720 kPa (90 psig); about 1,130 kPa (150 psig); or about 2,160 kPa (300 psig). In one or more embodiments, the steam introduced via line 145 can be superheated having a minimum superheat of about 15° C. (30° F.); about 30° C. (60° F.); about 45° C. (90° F.); about 60° C. (120° F.); or about 90° C. (150° F.).

The one or more catalysts supplied via line 115 can include catalysts useful for catalytically cracking the first hydrocarbon feed to provide one or more hydrocarbon products including, but not limited to ethylene, propylene and propane, mixed butanes and butylenes, naphthas, light cycle oil, heavy cycle oil, or any combination thereof The one or more catalysts can include, but are not limited to, one or more of the following: ZSM-11, ZSM-12, ZSM-23, ZSM-35, ZSM-38, Y-type zeolites, metal impregnated catalysts, zeolites, faujasite zeolites, modified faujasite zeolites, Y-type zeolites, ultrastable Y-type zeolites (USY), rare earth exchanged Y-type zeolites (REY), rare earth exchanged ultrastable Y-type zeolites (REUSY), rare earth free Z-21, Socony Mobil #5 zeolite (ZSM-5), high activity zeolite catalysts, mixtures thereof or combinations thereof.

The catalyst supplied via line 115 can be introduced to the riser reactor 105 at a rate proportionate to the first hydrocarbon feed. In one or more embodiments, the catalyst feed-to-hydrocarbon feed weight ratio can range from a minimum of about 4:1; about 6:1; or about 8:1 to a maximum of about 10:1; about 14:1; about 18:1; or about 30:1. In one or more specific embodiments the catalyst feed-to-hydrocarbon feed weight ratio in the riser reactor 105 can be about 4:1 to about 10:1. In order to maintain the desired mixture temperature of the first feed and catalyst, the one or more catalysts can be introduced to the riser reactor 105 at elevated temperature. In one or more embodiments, the temperature of the one or more catalysts can range from about 500° C. to about 900° C.; about 575° C. to about 875° C.; or about 650° C. (1,200° F.) to about 815° C. (1,500° F.). The combined first hydrocarbon feed and one or more catalysts (“first mixture”) can be maintained at a temperature (“first temperature”) of from about 450° C. (850° F.) to about 520° C. (970° F.); about 520° C. (970° F.) to about 540° C. (1,000° F.); or about 540° C. (1,000° F.) to about 565° C. (1,050° F.).

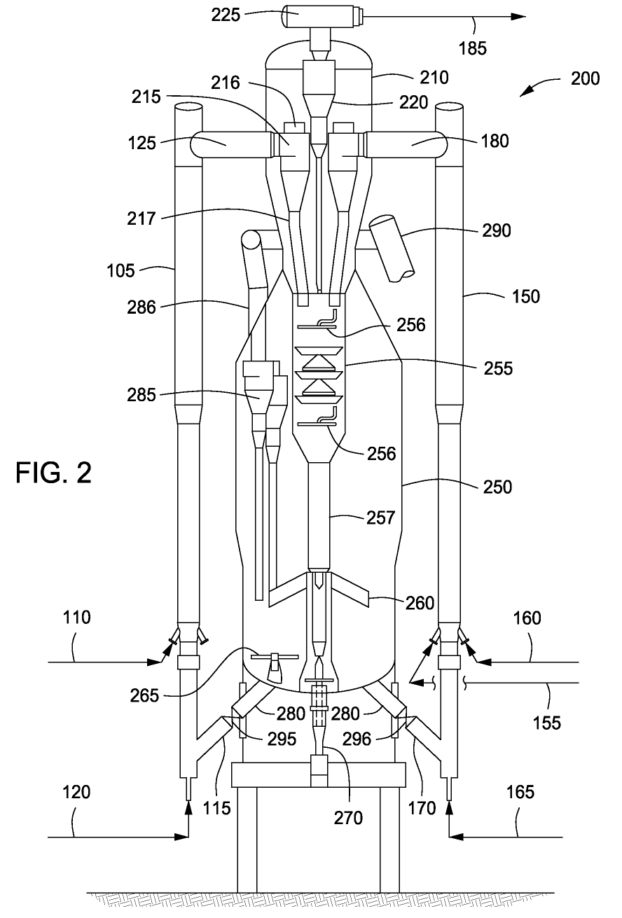

The riser reactor 105 can include any system, device or combination of systems or devices suitable for the cracking of one or more hydrocarbon feeds in the presence of one or more catalysts. The riser reactor 105 can include one or more risers on a fluidized catalytic cracker (“FCC”) described in greater detail with reference to FIG. 2. The riser reactor 105 can be configured in any physical orientation or geometry, including horizontal (0° elevation), vertical (90° elevation) including any intermediate angle therebetween. The riser reactor 105 can operate at a temperature of from about 450° C. (850° F.) to about 520° C. (970° F.); about 520° C. (970° F.) to about 540° C. (1,000° F.); or about 540° C. (1,000° F.) to about 565° C. (1,050° F.). The riser reactor 105 can operate at a pressure of from about 140 kPa (5 psig) to about 2,160 kPa (300 psig); about 140 kPa (5 psig) to about 1,130 kPa (150 psig); or from about 140 kPa (5 psig) to about 720 kPa (90 psig).

Within the riser reactor 105, in the presence of the one or more catalysts and at low severity conditions, the first hydrocarbon feed can crack, react, convert, and/or otherwise recombine to provide a mixture containing one or more cracked hydrocarbons (“first cracked mixture”). As the hydrocarbons present in the riser reactor 105 crack and decompose to form finished products, at least a portion of the first hydrocarbon feed can deposit as a layer of carbonaceous coke on the exterior surface of the one or more catalysts. The deposition of coke on the surface of the catalyst deactivates the catalyst and forms coke-covered catalyst. The coke-covered catalyst can exit the riser reactor 105 suspended in the cracked mixture in line 125.

In one or more embodiments, the hydrocarbons present in the first cracked mixture in line 125 can include one or more cycle oils. In one or more embodiments, the first cracked mixture in line 125 can have a light cycle oil concentration of from about 1% vol. to about 70% vol.; about 2% vol. to about 60% vol.; or from about 5% vol. to about 50% vol. In one or more embodiments, the first cracked mixture in line 125 can have a heavy cycle oil concentration of from about 1% vol. to about 70% vol.; about 2% vol. to about 60% vol.; or from about 5% vol. to about 50% vol. In one or more embodiments, the first cracked mixture in line 125 can have a solids concentration of from about 500 ppmw to about 50% wt.; about 2,500 ppmw to about 50% wt.; about 1% wt. to about 50% wt.; or from about 5% wt. to about 50% wt.

In one or more embodiments, one or more hydrocarbon feeds can be supplied to a second riser reactor 150. In one or more embodiments, a second hydrocarbon feed via line 155, a third hydrocarbon feed via line 160, the one or more catalysts via line 170 and, optionally, steam via line 165 can be introduced to the second riser reactor 150. In one or more embodiments, the second hydrocarbon feed in line 155 can include, but is not limited to one or more hydrocarbons having from four to twelve carbon atoms (C₄ to C₂₀), including one or more naphthas. In one or more specific embodiments, the second hydrocarbon feed in line 155 can include, but is not limited to one or more hydrocarbon compounds having from four to eight carbon atoms (C₄ to C₈). In one or more embodiments, the second hydrocarbon feed can include, but is not limited to one or more naphthas, olefins, liquefied petroleum gases or any combination thereof. In one or more embodiments, the second hydrocarbon feed can include, but is not limited to one or more C₄ hydrocarbons, one or more light naphthas, one or more straight naphthas, one or more straight run kerosene/diesel products, or any combination thereof In one or more embodiments, all or a portion of the second hydrocarbon feed can include one or more recycle products from the cracking of the first hydrocarbon feed, for example one or more light naphthas. The second hydrocarbon feed can have a Research Octane Number of from about 60 to about 88; about 65 to about 88; or about 70 to about 88. The second hydrocarbon feed can have a normal bulk boiling point of from about 95° C. (200° F.) to about 260° C. (500° F.); about 120° C. (250° F.) to about 240° C. (465° F.); or about 150° C. (300° F.) to about 220° C. (430° F.).

In one or more embodiments, the second hydrocarbon feed in line 155 can be partially or completely vaporized prior to introduction to the riser reactor 150. In one or more embodiments, the second hydrocarbon feed in line 155 can be about 25% wt. or more; about 50% wt. or more; about 75% wt. or more; about 90% wt. or more; about 95% wt. or more; about 99% wt. or more; or about 99.9% wt. or more vaporized prior to introduction to the riser reactor 150. The second hydrocarbon feed in line 155 can be introduced to the riser reactor 150 at ambient temperature or at an elevated temperature. In one or more embodiments, the temperature of the second hydrocarbon feed in line 155 can be a minimum of about 40° C. (105° F.); about 65° C. (150° F.); about 80° C. (175° F.); about 100° C. (210° F.); about 125° C. (260° F.); about 260° C. (500° F.); about 370° C. (700° F.); or about 480° C. (900° F.).

The optional steam introduced via line 165 can be either saturated or superheated. In one or more embodiments, the steam introduced via line 165 can be saturated, having a minimum supply pressure of about 135 kPa (5 psig); about 310 kPa (30 psig); about 510 kPa (60 psig); about 720 kPa (90 psig); about 1,130 kPa (150 psig); or about 2,160 kPa (300 psig). In one or more embodiments, the steam introduced via line 165 can be superheated having a minimum superheat of about 15° C. (30° F.); about 30° C. (60° F.); about 45° C. (90° F.); about 60° C. (120° F.); or about 90° C. (150° F.).

The one or more catalysts supplied via line 170 to the second riser reactor 150 can be the same as or different than the one or more catalysts supplied to the first riser reactor 105 via line 115. The one or more catalysts supplied via line 170 can include one or more catalysts suitable for catalytically cracking the second and third hydrocarbon feeds to provide one or more finished products including, but not limited to one or more olefinic hydrocarbons, one or more mixed hydrocarbons, or any combination thereof.

The catalyst can be supplied via line 170 to the one or more riser reactors 150 at a rate proportionate to the second hydrocarbon feed, the third hydrocarbon feed, or the combined second and third hydrocarbon feeds. In one or more embodiments, the catalyst feed-to-hydrocarbon feed weight ratio can range from a minimum of about 4:1; about 7:1; or about 10:1 to a maximum of about 18:1; about 25;1; about 30:1; or about 70:1. In one or more specific embodiments the catalyst feed-to-hydrocarbon feed weight ratio in the one or more riser reactors 150 can be about 7:1 to about 30:1. In one or more embodiments, the one or more catalysts can be introduced to the one or more riser reactors 150 at an elevated temperature to maintain the desired temperature if the hydrocarbon and catalyst mixture. In one or more embodiments, the temperature of the one or more catalysts in line 170 can range from about 500° C. to about 900° C.; about 575° C. to about 875° C.; or about 650° C. (1,200° F.) to about 815° C. (1,500° F.). The combined second hydrocarbon feed and one or more catalysts (“first mixture”) can be maintained at a temperature (“second temperature”) of from about 520° C. (970° F.) to about 565° C. (1,050° F.); or from about 540° C. (1,000° F.) to about 700° C. (1,290° F.).

The one or more riser reactors 150 can include any system, device or combination of systems or devices suitable for the cracking of one or more hydrocarbon feeds in the presence of the one or more catalysts. In one or more embodiments, the one or more riser reactors 150 can include one or more risers on a fluidized catalytic cracker (“FCC”) described in greater detail with reference to FIG. 2. In one or more embodiments, the one or more riser reactors 150 can include one or more risers on the same FCC as the one or more riser reactors 105. The one or more riser reactors 150 can be configured in any physical orientation or geometry, including horizontal (0° elevation), vertical (90° elevation) including any intermediate angle therebetween. The one or more riser reactors 150 can operate at the same or different temperature, pressure, and/or residence time than the one or more riser reactors 105. In one or more embodiments, the one or more riser reactors 150 can operate at a temperature of from about 520° C. (970° F.) to about 565° C. (1,050° F.); or from about 540° C. (1,000° F.) to about 700° C. (1,290° F.). The one or more riser reactors 150 can operate at a pressure of from about 140 kPa (5 psig) to about 2,160 kPa (300 psig); about 140 kPa (5 psig) to about 1,130 kPa (150 psig); or from about 140 kPa (5 psig) to about 720 kPa (90 psig).

The third hydrocarbon feed supplied via line 160 can be introduced at any point within the one or more riser reactors 150. In one or more embodiments, the third hydrocarbon feed in line 160 can be introduced at same point as the second hydrocarbon feed in line 155. In one or more embodiments, the third hydrocarbon feed in line 160 can be introduced at subsequent point to the second hydrocarbon feed in line 155. In one or more embodiments, the third hydrocarbon feed in line 160 can be introduced simultaneously, sequentially, alternatively or in any other manner or frequency in relation to the second hydrocarbon feed in line 155.

The third hydrocarbon feed in line 160 can be partially or completely vaporized prior to introduction to the one or more riser reactors 150. In one or more embodiments, the third hydrocarbon feed in line 160 can be about 5% wt. or more; about 10% wt. or more; about 25% wt. or more; about 50% wt. or more; about 75% wt. or more; about 90% wt. or more; or about 99.9% wt. or more vaporized prior to introduction to the one or more riser reactors 150. The third hydrocarbon feed in line 160 can be introduced to the one or more riser reactors 150 at ambient or elevated temperature. In one or more embodiments, the third hydrocarbon feed in line 160 can be at a temperature of about 40° C. (105° F.) or more; about 150° C. (300° F.) or more about 260° C. (500° F.) or more; or about 370° C. (700° F.) or more.

In one or more embodiments, the coke produced by the third hydrocarbon feed in line 160 can be sufficient to maintain the temperature (“third temperature”) of either or both the first feed and catalyst mixture within the first riser reactor 105 and the second feed and catalyst mixture within the second riser reactor 150. Lower severity operating conditions within the first riser 105 can increase the need for additional coke to provide adequate heat for catalyst regeneration. All or a portion of the additional coke can be produced using the second riser 150, which can be operated at higher severity conditions favoring the production of coke. In one or more embodiments, operating the first riser 105 at higher severity conditions can reduce or eliminate the need for the third hydrocarbon feed. In one or more embodiments, operating the first riser 105 at higher severity conditions can provide all or a portion of the heat necessary to process alternate feed stocks in the second riser 150, for example one or more imported hydrocarbons. In one or more embodiments, fuel can be fed continuously into the catalyst regenerator to supplement or replace the third hydrocarbon feed.

Within the one or more riser reactors 150, the second hydrocarbon feed and third hydrocarbon feed, and the one or more catalysts can crack, react, convert, and/or otherwise recombine to provide a mixture containing one or more cracked hydrocarbons (“second cracked mixture”). The hydrocarbons present in the one or more riser reactors 150 can crack and convert to provide one or more finished products, at least a portion of the second and third hydrocarbon feeds can deposit as a layer of carbonaceous coke on the exterior surface of the one or more catalysts present in the one or more riser reactors 150. The deposition of coke on the surface of the one or more catalysts can deactivate the catalyst, and can form coke-covered catalyst. The coke-covered catalyst can exit the one or more riser reactors 150 suspended in the second cracked mixture in line 180.

The hydrocarbons present in the second cracked mixture in line 180 can include one or more olefinic hydrocarbons, such as ethylene and propylene, and one or more mixed hydrocarbons. The one or more mixed hydrocarbons present in the second cracked mixture in line 180 can have a Research Octane Number greater than the Research Octane Number of the second hydrocarbon feed in line 155.

In one or more embodiments, the second cracked mixture in line 180 can have an ethylene concentration of from about 0.1% vol. to about 20% vol.; about 0.5% vol. to about 17% vol.; or from about 1% vol. to about 15% vol. In one or more embodiments, the second cracked mixture in line 180 can have a propylene concentration of from about 0.1% vol. to about 20% vol.; about 0.5% vol. to about 17% vol.; or from about 1% vol. to about 15% vol. In one or more embodiments, in the second cracked mixture in line 180 can have a mixed hydrocarbons concentration of from about 1% vol. to about 50% vol.; about 5% vol. to about 40% vol.; or from about 5% vol. to about 30% vol. In one or more embodiments, the second cracked mixture in line 180 can have a solids concentration of from about 500 ppmw to about 50% wt.; about 2,500 ppmw to about 75% wt.; about 1% wt. to about 98% wt.; or from about 5% wt. to about 99% wt.

In one or more embodiments, all or a portion of the mixed hydrocarbons in the second cracked mixture in line 180 can be useful in providing a high octane gasoline blendstock. In one or more embodiments, all or a portion of the mixed hydrocarbons in the second cracked mixture in line 180 can have a Research Octane Number of from about 88 to about 100; about 88 to about 97; or about 88 to about 95. In one or more embodiments, all or a portion of the mixed hydrocarbons in the second cracked mixture in line 180 can have a bulk normal boiling point of from about 95° C. (200° F.) to about 260° C. (500° F.); about 120° C. (250° F.) to about 240° C. (465° F.); or about 150° C. (300° F.) to about 220° C. (430° F.).

The first cracked mixture in line 125 and the second cracked mixture in line 180 can be combined to provide a third mixture via line 185. The third mixture in line 185 can contain light cycle oil, heavy cycle oil, one or more light hydrocarbons containing twelve or fewer carbon atoms, one or more olefinic hydrocarbons, one or more mixed hydrocarbons, mixtures thereof, derivatives thereof, or any combination thereof In one or more embodiments, the third mixture in line 185 can have an ethylene concentration of from about 0.5% vol. to about 40% vol; about 1% vol. to about 30% vol.; or about 2% vol. to about 25% vol. In one or more embodiments, the third mixture in line 185 can have a propylene concentration of from about 0.5% vol. to about 40% vol; about 1% vol. to about 30% vol.; or about 2% vol. to about 25% vol. In one or more embodiments, the third mixture in line 185 can have a mixed hydrocarbon concentration of from about 1% vol. to about 80% vol; about 2% vol. to about 60% vol.; or about 3% vol. to about 30% vol. In one or more embodiments, the third mixture in line 185 can have a light cycle oil concentration of from about 1% vol. to about 50% vol; about 2% vol. to about 40% vol.; or about 3% vol. to about 30% vol. In one or more embodiments, the third mixture in line 185 can have a temperature of from about 200° C. (390° F.) to about 1,700° C. (3,090° F.); about 300° C. (570° F.) to about 1,400° C. (2,550° F.); about 400° C. (750° F.) to about 1,000° C. (1,830° F.); or about 500° C. (930° F.) to about 700° C. (1,290° F. In one or more embodiments, the third mixture in line 185 can have a pressure of from about 140 kPa (5 psig) to about 2,160 kPa (300 psig); about 140 kPa (5 psig) to about 1,130 kPa (150 psig); or from about 140 kPa (5 psig) to about 720 kPa (90 psig).

In one or more embodiments, at least a portion of the one or more light hydrocarbons present in the third mixture, such as one or more hydrocarbons in the gasoline boiling range, can be selectively separated and recycled to provide at least a portion of the second hydrocarbon feed in line 155. In one or more embodiments, at least a portion of the one or more mixed hydrocarbons can be selectively separated from the third mixture for use as a high-octane gasoline blendstock. In one or more embodiments, at least a portion of the light cycle oils can be selectively separated from the third mixture to provide a diesel fuel blendstock.

FIG. 2 depicts another illustrative system 200 for upgrading one or more hydrocarbons according to one or more embodiments. The system 200 can include two or more riser reactors 105, 150, one or more disengagers 210, and one or more regenerators 250. The one or more disengagers 200 can include one or more riser cyclones 215, one or more upper cyclones 220, one or more plenums 225, one or more catalyst strippers 255, one or more catalyst distributors 260, and one or more plug valves 270. The one or more regenerators 250 can include one or more air distributors 265, one or more regenerator cyclones 285, and one or more plenums 290. In one or more embodiments, the system 200 can include, but is not limited to one or more fluidized catalytic cracking systems 200 as depicted in FIG. 2.

The first cracked mixture can exit the first riser reactor 105 via line 125 and can be introduced to the one or more disengagers 210. Similarly, the second cracked mixture can exit the second riser reactor 150 via line 180 and enter the one or more disengagers 210. Within the disengager 210, the first cracked mixture and the second cracked mixture can flow into the one or more riser cyclones 215 wherein at least a portion of the coke-covered catalyst present in the first cracked mixture and the second cracked mixture can be selectively separated. The first cracked mixture and the second cracked mixture can exit the one or more riser cyclones 215 via one or more discharges 216, flowing into the disengager 210. Within the disengager 210, the first cracked mixture and the second cracked mixture can mix and combine to form the third mixture. The third mixture can flow into the one or more upper cyclones 220 wherein at least a portion of the coke-covered catalyst present in the third mixture can be selectively separated to provide a near-solids free third mixture via line 185. As used herein, the term “near-solids free” can include fluids having a solids concentration of from about 5 ppmw to about 5% wt.; about 10 ppmw to about 4% wt.; about 25 ppmw to about 3.5% wt.; or from about 50 ppmw to about 3% wt.

The near solids-free third mixture can flow from the one or more upper cyclones 220 into the one or more disengager plenums 225 for withdrawal and subsequent fractionation or separation into one or more finished hydrocarbon products, for example one or more light cycle oils, one or more olefinic hydrocarbons and/or one or more mixed hydrocarbons in the gasoline boiling range having a Research Octane Number of about 88 or more.

The coke-covered catalyst separated exiting the one or more riser cyclones 215 and the one or more upper cyclones 220 can flow through the disengager 210 and enter the one or more catalyst strippers 255. Steam can optionally be sparged through the coke-covered catalyst within the one or more catalyst strippers 255 using one or more steam distributors 256 (two such steam distributors 256 are depicted in FIG. 2). The passage of steam through the catalyst stripper 255 can assist in removing residual hydrocarbons entrained or entrapped within the coke-covered catalyst prior to regenerating the catalyst within the one or more regenerators 250. The steam, carrying one or more hydrocarbons stripped from the coke-covered catalyst in the catalyst stripper 255, can flow into the disengager 210, thence into the one or more upper cyclones 220.

The steam supplied to the catalyst stripper 255 via the one or more distributors 256 can be saturated or superheated. In one or more embodiments, the steam introduced via the one or more distributors 256 can be saturated, having a minimum supply pressure of about 135 kPa (5 psig); about 310 kPa (30 psig); about 510 kPa (60 psig); about 720 kPa (90 psig); about 1,130 kPa (150 psig); or about 2,160 kPa (300 psig). In one or more embodiments, the steam introduced via the one or more distributors 256 can be superheated having a minimum superheat of about 15° C. (30° F.); about 30° C. (60° F.); about 45° C. (90° F.); about 60° C. (120° F.); or about 90° C. (150° F.).

Coke-covered catalyst can flow from the catalyst stripper 255 into one or more standpipes 257. A first portion of the coke-covered catalyst within the one or more standpipes 257 can flow through one or more distributors 260 into the one or more regenerators 250. The remaining portion of coke-covered catalyst within the standpipe 257 can be withdrawn from the one or more standpipes 257 using one or more plug valves 270. In one or more embodiments, about 5% wt. or more; about 10% wt. or more; about 25% wt. or more; about 50% wt. or more; about 75% wt. or more; about 85% wt. or more; about 90% wt. or more; about 95% wt. or more; or about 99% wt. or more of the coke-covered catalyst in the standpipe 257 can pass through the one or more distributors 260 into the one or more regenerators 250 with the balance withdrawn from the standpipe 257 using the one or more plug valves 270. At least a portion of one or more fresh catalysts can be added to the system 200 via either or both of the one or more riser reactors 105, 150 and/or the one or more regenerators 250.

Within the one or more regenerators 250 one or more oxidants can be distributed using one or more distributors 265. The addition of one or more oxidants to the coke-covered catalyst within the one or more regenerators 250 can result in the partial or complete oxidation and/or combustion of the coke on the surface of the one or more catalysts into one or more waste gases including, but not limited to carbon monoxide, carbon dioxide, hydrogen, water vapor, mixtures thereof or combinations thereof. The removal of the coke from the surface of the one or more catalysts can re-expose the surface of the catalyst, thereby providing one or more reactivated and/or regenerated catalysts. At least a portion of the one or more reactivated and/or regenerated catalysts can be recycled from the one or more regenerators 250 to the one or more riser reactors 105, 150 via one or more standpipes 280. In one or more embodiments, one or more standpipes 280 can discharge through valve 295 into the catalyst feed line 135 to the one or more riser reactors 105. In one or more embodiments, one or more standpipes 280 can discharge through valve 296 into the catalyst feed line 170 to the one or more riser reactors 150.

As used herein, the term “oxidants” can refer to any substance, mixture, compound or element suitable for oxidizing the coke present on the surface of the one or more catalysts. Oxidants can include, but are not limited to, air, oxygen enriched air (air having an oxygen concentration greater than 21% wt.), oxygen, or nitrogen enriched air (air having a nitrogen concentration greater than 79% wt.).

At least a portion of the catalyst requirement of the one or more riser reactors 105 can be satisfied using regenerated catalyst from the regenerator 250. Similarly, at least a portion of the catalyst requirement of the one or more riser reactors 150 can be satisfied using regenerated catalyst from the regenerator 250. In one or more embodiments, about 25% wt. or more; about 50% wt. or more; about 75% wt. or more; about 85% wt. or more; about 90% wt. or more; about 95% wt. or more of the catalyst in the one or more riser reactors 105 can be supplied using regenerated catalyst from the regenerator 250. In one or more embodiments, about 25% wt. or more; about 50% wt. or more; about 75% wt. or more; about 85% wt. or more; about 90% wt. or more; about 95% wt. or more of the catalyst in the one or more riser reactors 150 can be supplied using regenerated catalyst from the regenerator 250.

The one or more waste gases generated by the oxidation and/or combustion of the coke on the surface of the catalyst in the one or more regenerators 250 can flow into the one or more regenerator cyclones 285 wherein at least a portion of the catalyst can be removed and returned to the one or more regenerators 250. Waste gas from the one or more regenerator cyclones 285 can exit the regenerator via one or more ducts 286. The waste gas can be collected in the one or more regenerator plenums 290. The collected waste gases in the one or more regenerator plenums 290 can be directed for subsequent recovery, reuse, recycle, treatment, and/or disposal.

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1) A method for upgrading one or more hydrocarbons comprising:
cracking a first hydrocarbon in the presence of one or more catalysts at a first temperature to provide a first cracked mixture comprising one or more light cycle oils and a first coked catalyst;
mixing a second hydrocarbon comprising one or more C4 to C20 hydrocarbons having a Research Octane Number of less than 88 with the one or more catalysts to provide a first mixture at a second temperature;
introducing a third hydrocarbon to provide additional coke on the first coked catalyst;
cracking a second hydrocarbon in the presence of the one or more catalysts at a second temperature to provide a second cracked mixture comprising propylene and one or more hydrocarbons having a Research Octane Number of greater than 88, and a second coked catalyst; and
combining the first cracked mixture and the second cracked mixture to provide a third mixture comprising the one or more light cycle oils, the propylene, the one or more hydrocarbons having a Research Octane Number greater than about 88, the first coked catalyst and the second coked catalyst.

2) The method of claim 1, further comprising:
selectively separating the first coked catalyst and the second coked catalyst from the third mixture to provide a separated coked catalyst and a product mixture;
regenerating at least a portion of the separated coked catalyst to provide at least a portion of the one or more catalysts; and
selectively separating the product mixture to provide one or more finished products comprising one or more olefinic hydrocarbons, one or more naphthas having a Research Octane Number greater than about 88, and one or more light cycle oils.

3) The method of claim 2 wherein fuel is added to the catalyst regeneration step to maintain an acceptable temperature for catalyst regeneration.

4) The method of claim 3 wherein at least a portion of the fuel comprises all or a portion of the one or more finished products.

5) The method of claim 2 wherein at least a portion of the light cycle oil comprises the at least a portion of the third hydrocarbon.

6) The method of claim 2 wherein the third hydrocarbon comprises demetallized oils, deasphalted oils, vacuum gas oils, fuel oils, gas oils, resids having twenty or more carbon atoms (C20+), mixtures thereof, or any combination thereof.

7) The method of claim 1 wherein the third hydrocarbon is added to the first hydrocarbon prior to mixing the first hydrocarbon with the one or more catalysts.

8) The method of claim 1 wherein the third hydrocarbon is added after partially cracking the first hydrocarbon in the presence of the one or more catalysts

9) The method of claim 1 wherein at least a portion of the first hydrocarbon comprises at least a portion of the third hydrocarbon.

10) The method of claim 1 wherein the difference between the second temperature and the third temperature is 165° C. or less.

11) The method of claim 1 wherein the difference between the first temperature and the third temperature is 110° C. or more.

12) The method of claim 1 wherein at least a portion of the first mixture is cracked at the second temperature prior to the addition of the third hydrocarbon.

13) The method of claim 1 wherein the first temperature is about 450° C. to about 565° C.

14) The method of claim 1 wherein the second temperature is about 520° C. to about 700° C.

15) The method of claim 1 wherein the catalyst-to-first hydrocarbon weight ratio is about 4:1 to about 30:1.

16) The method of claim 1 wherein the catalyst-to-second hydrocarbon weight ratio is about 7:1 to about 70:1.

17) The method of claim 1 wherein at least a portion of the second hydrocarbon is vaporized prior to mixing with the one or more catalysts.

18) The method of claim 1 wherein the first hydrocarbon comprises demetallized oils, deasphalted oils, vacuum gas oils, fuel oils, gas oils, resids having twenty or more carbon atoms (C20+), mixtures thereof or any combination thereof.

19) The method of claim 1 wherein the one or more catalysts comprise one or more of the following: ZSM-11, ZSM-12, ZSM-23, ZSM-35, ZSM-38, Y-type zeolites, metal impregnated catalysts, zeolites, faujasite zeolites, modified faujasite zeolites, Y-type zeolites, ultrastable Y-type zeolites (USY), rare earth exchanged Y-type zeolites (REY), rare earth exchanged ultrastable Y-type zeolites (REUSY), rare earth free Z-21, Socony Mobil #5 zeolite (ZSM-5), or high activity zeolite catalysts.

20) A method for upgrading one or more hydrocarbons comprising:
cracking a first hydrocarbon in the presence of one or more catalysts at a first temperature to provide first cracked mixture comprising one or more light cycle oils and a first coked catalyst;
mixing a second hydrocarbon comprising one or more C4 to C20 hydrocarbons having a Research Octane Number of less than 88 with the one or more catalysts to provide a first mixture at a second temperature;
cracking a second hydrocarbon in the presence of the one or more catalysts at a second temperature to provide a second cracked mixture comprising propylene and one or more hydrocarbons having a Research Octane Number of greater than 88, and a second coked catalyst;
introducing a third hydrocarbon to provide additional coke on the second coked catalyst; and
combining the first cracked mixture and the second cracked mixture to provide a third mixture comprising the one or more light cycle oils, the propylene, the one or more hydrocarbons having a Research Octane Number greater than about 88, the first coked catalyst and the second coked catalyst.
